(19) United States
(12) Patent Application Publication
(10) Pub. No.: US 2022/0346654 A1
(43) Pub. Date: Nov. 3, 2022

(54) PORTABLE ELECTROCARDIOGRAPHIC DEVICE

(71) Applicant: OMRON HEALTHCARE Co., Ltd., Kyoto (JP)

(72) Inventors: Mika EZOE, Kyoto (JP); Mitsuru SAMEJIMA, Kyoto (JP); Shinya KODAKA, Kyoto (JP)

(21) Appl. No.: 17/663,617

(22) Filed: May 16, 2022

Related U.S. Application Data
(63) Continuation of application No. PCT/JP2020/041107, filed on Nov. 2, 2020.

(30) Foreign Application Priority Data
Nov. 20, 2019 (JP) 2019-209992

(51) Int. Cl. A61B 5/0245; A61B 5/1172; A61B 5/00; A61B 5/28

(52) U.S. Cl. CPC A61B 5/0245; A61B 5/1172; A61B 5/7475; A61B 5/743; A61B 5/6826; A61B 5/683; A61B 5/28

(57) ABSTRACT

A portable electrocardiographic device includes: an electrode unit configured to detect an electrocardiographic waveform; and a control unit configured to record, in a storage unit, the electrocardiographic waveform detected by the electrode unit. The electrode unit includes a pair of measurement electrodes for measuring the electrocardiographic waveform, and an earth electrode for detecting a reference potential for a change in a potential of a body, and the earth electrode is disposed in such a manner that a distal end side of a finger of a subject that is a target of the electrocardiographic waveform measurement is brought in contact with the earth electrode, and one of the measurement electrodes is disposed on a proximal end side of the finger of the subject, the distal end side of which is in contact with the earth electrode.

### CROSS-REFERENCE TO RELATED APPLICATIONS

This application is the U.S. national stage application filed pursuant to 35 U.S.C. 365(c) and 120 as a continuation of International Patent Application No. PCT/JP2020/041107, filed Nov. 2, 2020, which application claims priority to Japanese Patent Application No. 2019-209992, filed Nov. 20, 2019, which applications are incorporated herein by reference in their entireties.

### TECHNICAL FIELD

The present invention relates to a portable electrocardiographic device enabling measurement of electrocardiographic waveforms in daily life or the like.

### BACKGROUND ART

A portable electrocardiographic measurement device (hereinafter, also referred to as “portable electrocardiographic device”) has been proposed and known that enables immediate measurement of an electrocardiographic waveform, upon occurrence of abnormality such as chest pain and palpitation in daily life. When symptoms such as palpitation occur at home, outside the home, or the like, doctors or the like can detect a heart disease at an early timing and perform appropriate treatment based on data on the electrocardiographic waveform measured by the electrocardiographic device.

Prior art documents describing techniques related to the one described in the present specification include the following patent documents: Patent Document 1: JP 2005-000420A; Patent Document 2: JP 2005-211388A; Patent Document 3: JP 4443335B; and, Patent Document 4: U.S. Pat. No. 9,089,270.

### SUMMARY OF INVENTION

### Technical Problem

FIG. 9(a) and FIG. 9(b) illustrate a known example case of electrocardiographic measurement using a portable electrocardiographic measurement device according to Patent Document 2. According to the disclosure, the right hand forefinger is placed in such a manner that the fingertip is brought into contact with a fingertip side electrode and the middle phalanx is brought into contact with a middle phalanx side electrode. As illustrated in FIG. 9(b), Patent Document 2 discloses a known example of a structure in which the fingertip side electrode serves as a negative electrode 121, that is, a measurement electrode, and the middle phalanx side electrode serves as an indifferent electrode 123, that is, an earth electrode, with a positive electrode 122 provided on the opposite side. The measurement electrode described above is provided on the fingertip side where force is likely to be applied and movement is likely to occur. Thus, during the electrocardiographic measurement, the measurement accuracy of the electrocardiographic waveform might be compromised due to an instable state of contact with the measurement electrode.

The present invention has been made in view of the problems described above, and an object of the present invention is to provide a technique of a portable electrocardiographic device with which usability as well as accuracy of electrocardiographic waveform measurement in daily life can be improved.

### Solution to Problem

A portable electrocardiographic device according to the present invention for solving the problem described above includes:

an electrode unit configured to detect an electrocardiographic waveform; and
a control unit configured to record in a storage unit, the electrocardiographic waveform detected by the electrode unit, in which the electrode unit includes a pair of measurement electrodes for measuring the electrocardiographic waveform, and an earth electrode for detecting a reference potential for a change in a potential of a body, and the earth electrode is disposed in such a manner that a distal end side of a finger of a subject that is a target of the electrocardiographic waveform measurement is brought in contact with the earth electrode, and one of the measurement electrodes is disposed on a proximal end side of the finger of the subject, the distal end side of which is in contact with the earth electrode.

With this configuration, the portable electrocardiographic device can be held during the electrocardiographic waveform measurement, with the distal end side, of the right hand forefinger bent in a substantially L shape along a curved shape on an upper portion side of a main body, being in close contact with the earth electrode in a hooked manner, and with the proximal end side of the finger being in close contact with one of the measurement electrodes. This means that the proximal end side of the finger that is less likely to move can be in close contact with the measurement electrode, whereby a stable contact state is achieved during the measurement. As a result, a stable contact resistance is achieved between the finger of the subject and the electrode, whereby the accuracy of the electrocardiographic waveform measurement can be improved.

In the present invention, the distal end side may include a distal phalanx of the finger of the subject that is the target of the electrocardiographic waveform measurement, and the proximal end side may include a middle phalanx site of the finger, the distal end side of which is in contact with the earth electrode. With this configuration, the measurement can be performed despite a difference in physical characteristics, such as a difference in the length of the finger between users that are the target of the electrocardiographic waveform measurement using the portable electrocardiographic device.

In the present invention, the earth electrode disposed in such a manner that the distal end side of the finger of the subject is brought in contact with the earth electrode may be provided with a fingerprint detection unit configured to detect a fingerprint of the finger in contact with the fingerprint detection unit. With this configuration, whether the contact state of the fingertip in contact with the earth electrode is appropriate can be determined based on the fingerprint information on the subject detected by the fingerprint detection unit.

In the present invention, a region, of the earth electrode, where the fingerprint detection unit is provided may be formed by a transparent electrode. With this configuration, an optical scheme can be employed for a fingerprint comparison scheme for detecting a fingerprint of the fingertip in contact with the earth electrode, whereby a cost related to the portable electrocardiographic device can be reduced.

In the present invention, the earth electrode may be provided with a fingerprint detection window for detecting a fingerprint of the finger in contact with the earth electrode. With this configuration, a region, in the earth electrode, for detecting the fingerprint can be confirmed through visual confirmation or the like, whereby the usability at the time of electrocardiographic waveform measurement is improved.

In the present invention, the control unit may record, in the storage unit, fingerprint information on the subject detected by the fingerprint detection unit provided to the earth electrode, and determine whether a result of comparison between registered fingerprint information registered in the storage unit and the fingerprint information on the subject satisfies a predetermined condition. With this configuration, the registered fingerprint information registered in advance can be compared with the fingerprint information on the subject detected by the fingerprint detection unit.

In the present invention, the control unit may start measuring the electrocardiographic waveform of the subject, when the result of the comparison between the registered fingerprint information registered in the storage unit and the fingerprint information on the subject satisfies the predetermined condition. With this configuration, the contact state of the fingertip in contact with the earth electrode can be determined to be appropriate, when the result of the comparison satisfies the predetermined condition.

In the present invention, the control unit may start measuring the electrocardiographic waveform of the subject, when the result of the comparison between the registered fingerprint information registered in the storage unit and the fingerprint information on the subject satisfies the predetermined condition. With this configuration, the contact state of the fingertip in contact with the earth electrode can be determined to be appropriate, when the result of the comparison satisfies the predetermined condition.

In the present invention, when the result of the comparison between the registered fingerprint information registered in the storage unit and the fingerprint information on the subject is determined not to satisfy the predetermined condition, the subject is notified of fingerprint authentication using the fingerprint information on the subject having failed, or a contact state of the finger in contact with the earth electrode being inappropriate. With this configuration, the subject can be notified of the finger being wrongly brought in contact with the earth electrode. Furthermore, the subject can be prompted to perform the electrocardiographic waveform measurement under an appropriate contact state. With the portable electrocardiographic device, the usability at the time of electrocardiographic waveform measurement can be improved.

### Advantageous Effects of Invention

The present invention can provide a technique of a portable electrocardiographic device with which usability as well as accuracy of electrocardiographic waveform measurement in daily life can be improved.

### BRIEF DESCRIPTION OF DRAWINGS

FIG. 1 is a diagram illustrating an example of a configuration of a portable electrocardiographic device according to a first embodiment.

FIG. 2 is a diagram illustrating an example of a system configuration of the portable electrocardiographic device according to the first embodiment.

FIG. 3 is a flowchart illustrating an example of electrocardiographic waveform measurement processing in the portable electrocardiographic device according to the first embodiment.

FIG. 4(a) illustrates an example of a graph of an electrocardiographic waveform measured with an insufficient state of contact with the measurement electrode that is the electrode c 4.

FIG. 4(b) illustrates an example of a graph of an electrocardiographic waveform measured by the portable electrocardiographic device 1 according to the present embodiment.

FIG. 5 is a diagram illustrating an example of a configuration of a portable electrocardiographic device according to a second embodiment.

FIG. 6 is a diagram illustrating an example of a system configuration of the portable electrocardiographic device according to the second embodiment.

FIG. 7 is a flowchart illustrating an example of processing related to initial setting using fingerprint information in the portable electrocardiographic device according to the second embodiment.

FIG. 8 is a flowchart illustrating an example of processing related to electrocardiographic waveform measurement using fingerprint information in the portable electrocardiographic device according to the second embodiment.

FIG. 9 is a diagram illustrating a portable electrocardiographic measurement device according to a known example.

### DESCRIPTION OF EMBODIMENTS

Embodiments of the present invention will be specifically described below with reference to the drawings.

### First Embodiment

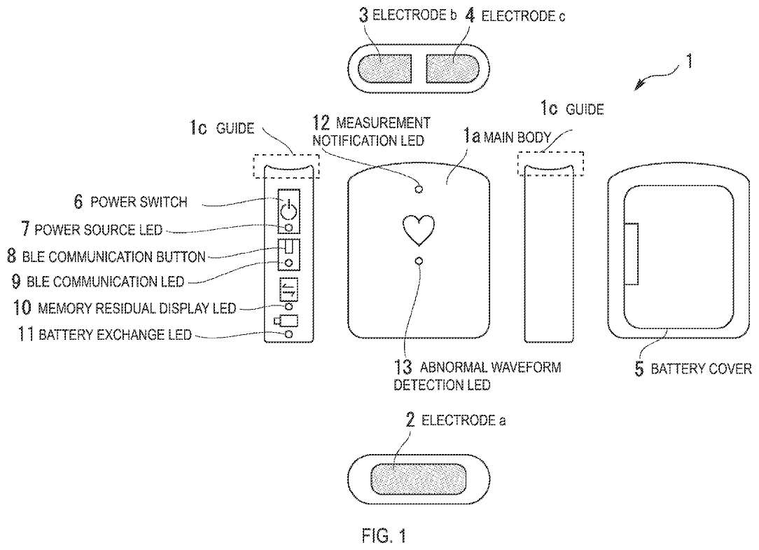

First, an example of the embodiments of the present invention will be described based on FIGS. 1 to 4. It should be noted that the dimension, material, shape, relative arrangement and the like of the components described in the present embodiment are not intended to limit the scope of this invention to them alone, unless otherwise stated.

Configuration of Portable Electrocardiographic Device

FIG. 1 is a diagram illustrating an example of a configuration of a portable electrocardiographic device according to the present embodiment. FIG. 1 illustrates an example of a six-view diagram of a portable electrocardiographic device 1. As illustrated in FIG. 1, the portable electrocardiographic device 1 includes an electrode a 2, an electrode b 3, and an electrode c 4 for measuring an electrocardiographic waveform of a subject. The portable electrocardiographic device 1 according to the present embodiment provides functions such as: measurement of an electrocardiographic waveform using the electrodes described above; notification display during the measurement; and detection of an abnormal waveform based on measurement data.

In FIG. 1, each electrode of the portable electrocardiographic device 1 is formed of a conductive member. The electrode a 2 is provided on a bottom portion side of a main body 1a. The electrode b 3 and the electrode c 4 are provided on an upper portion side of the main body 1a. The electrode b 3 is provided on the left side of the upper portion side of the main body 1a. The electrode c 4 is provided on the right side of the upper portion side of the main body 1a. The upper portion side of the main body 1a, where the electrode b 3 and the electrode c 4 are disposed, has a smoothly curved shape so that the right hand forefinger of the subject can easily come into contact with the upper portion side for example. The upper portion side of the main body 1a, where the electrode b 3 and the electrode c 4 are disposed, is processed to be in a recess form (guide 1c) to facilitate close contact of the right hand forefinger, as indicated by a rectangular region surrounded by a dashed line.

In the portable electrocardiographic device 1 according to the present embodiment, the electrode a 2 provided on the bottom portion of the main body 1a and the electrode c 4 provided on the right side of the upper portion of the main body 1a form a pair of measurement electrodes. Of the pair of measurement electrodes, the electrode a 2 serves as the positive electrode and the electrode c 4 serves as the negative electrode, for example. In the portable electrocardiographic device 1 according to the present embodiment, the electrode b 3 provided on the left side of the upper portion of the main body 1a is configured as an earth electrode for finding a reference potential for a change in potential of a body.

In a case where lead I measurement is performed for the electrocardiographic measurement, for example, the subject holds the portable electrocardiographic device 1 with his or her right hand, and brings the electrode a 2 provided on the bottom portion of the main body 1a in contact with his or her left palm. The forefinger of the right hand holding the portable electrocardiographic device 1 is brought in close contact with the electrode b 3 and the electrode c 4 along the recess (guide 1c) formed by the processing on the upper portion side of the main body 1a. For example, the electrocardiographic measurement is performed on the subject, with the electrode a 2, provided on the bottom portion, pressed in a direction to be pressed onto the left palm, from the upper portion side of the main body 1a where the electrode b 3 and the electrode c 4 are provided. The right hand can hold the portable electrocardiographic device 1 while pressing the portable electrocardiographic device 1 in the direction of pressing toward the left palm, with the right hand forefinger bent in substantially L shape along the curved shape on the upper portion side of the main body 1a to have the distal end side of the finger brought in close contact with the electrode b 3 on the left side in a hooked manner, and to have the proximal end side brought in close contact with the electrode c 4 on the right side. The “distal end side” brought in close contact with the electrode b 3 includes a site corresponding to the distal phalanx of the right hand forefinger. The “proximal end side” brought in close contact with the electrode c 4 includes a site corresponding to the middle phalanx part of the right hand forefinger, for example.

In a case where lead V4 measurement is performed for the electrocardiographic measurement, for example, the subject holds the portable electrocardiographic device 1 with his or her right hand, while bringing the electrode a 2 provided on the bottom portion of the main body 1a in contact with the skin on the lower side of the left nipple which is slightly on the left side of the epigastric region of the left chest. The forefinger of the right hand holding the portable electrocardiographic device 1 is brought in close contact with the electrode b 3 and the electrode c 4 along the recess (guide 1c) formed by the processing on the upper portion side of the main body 1a. The electrocardiographic measurement is performed, with the electrode a 2, provided on the bottom portion, pressed in a direction to be pressed onto a measurement site, from the upper portion side of the main body 1a where the electrode b 3 and the electrode c 4 are provided. Also in the case of the lead V4 measurement, the portable electrocardiographic device 1 can be held with the distal end side, of the right hand forefinger bent in a substantially L shape along the curved shape on the upper portion side of the main body 1a, brought in close contact with the electrode b 3 on the left side in a hooked manner, and with the proximal end side brought in contact with the electrode c 4 on the right side. Also in this measurement mode, the “distal end side” brought in close contact with the electrode b 3 includes a site corresponding to the distal phalanx of the right hand forefinger, and the “proximal end side” brought in close contact with the electrode c 4 includes a portion corresponding to the middle phalanx part of the right hand forefinger.

In the present embodiment, close contact between the measurement electrode and the earth electrode can be facilitated during the electrocardiographic measurement, with the electrode b 3 serving as the earth electrode disposed at the position to be in contact with the distal end side of the right hand forefinger, and with the electrode c 4 serving as the negative electrode of the measurement electrodes disposed at the position to be in contact with the proximal end side as described above. As a result, with the portable electrocardiographic device 1 according to the present embodiment, the instability of the contact state with the measurement electrode can be suppressed. Since superimposition of noise based on the instability of contact state and the like can be suppressed, the accuracy of the electrocardiographic waveform measurement can be improved.

The portable electrocardiographic device 1 has a left side surface provided with a power switch 6, a power source LED 7, and a Bluetooth (trade name) low energy (BLE) communication button 8 for performing BLE communications. Similarly, a BLE communication LED 9, a memory residual display LED 10, and a battery exchange LED 11 are provided. The power switch 6 is a switch that is pressed for turning on the portable electrocardiographic device 1, and the power source LED 7 is a light emitting element that is turned on when the power is turned on. The BLE communication button 8 is an operation part for executing function of communicating with a device supporting a BLE communication scheme, and the BLE communication LED 9 is a light emitting element that is turned on when the BLE communications are performed. The communication function of the portable electrocardiographic device 1 may be of a wireless communication method such as infrared communications or transmission of information using ultrasonic waves, or of a wired communication scheme implemented through connection using a cable, a connector, or the like. The memory residual display LED 10 is a light emitting element indicating a state of the remaining capacity of a memory unit 106 described below. The battery exchange LED 11 is a light emitting element that is turned on when the remaining power of the power source (battery) of the portable electrocardiographic device 1 falls below a predetermined value, to prompt the battery exchange.

The portable electrocardiographic device 1 has another side surface provided with a measurement notification LED 12 and an abnormal waveform detection LED 13. The measurement notification LED 12 is a light emitting element that is turned on or blinks during the electrocardiographic waveform measurement. The abnormal waveform detection LED 13 is a light emitting element that is turned on when an abnormal waveform is detected as the measured electrocardiographic waveform. With the abnormal waveform detection LED 13 turned on, the subject is notified of an abnormal waveform being detected from measurement data on the electrocardiographic waveform.

System Configuration of Portable Electrocardiographic Device

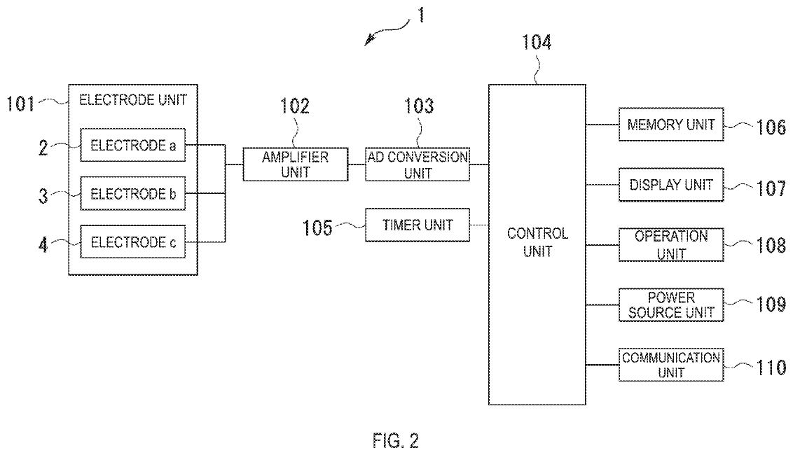

The system configuration of the portable electrocardiographic device is now described. FIG. 2 is a diagram illustrating an example of a system configuration of the portable electrocardiographic device 1 according to the present embodiment. As illustrated in FIG. 2, the portable electrocardiographic device 1 includes an electrode unit 101, an amplifier unit 102, an analog to digital (AD) conversion unit 103, a control unit 104, and a timer unit 105. The configuration of the portable electrocardiographic device 1 further includes the memory unit 106, a display unit 107, an operation unit 108, a power source unit 109, and a communication unit 110. The control unit 104, the timer unit 105, the memory unit 106, the display unit 107, the operation unit 108, the power source unit 109, and the communication unit 110 are connected to each other.

The electrode unit 101 includes the electrode a 2 and the electrode c 4 that function as a pair of measurement electrodes, and the electrode b 3 that functions as an earth electrode. With the electrode unit 101 brought in contact with the skin of the subject, an electrocardiographic waveform in a predetermined period is detected. The electrocardiographic waveforms detected by the electrodes of the electrode unit 101 are each input to the amplifier unit 102 connected to the electrode unit. The amplifier unit 102 amplifies a signal detected by the electrode unit 101, and outputs the resultant signal to the AD conversion unit 103. The AD conversion unit 103 performs digital conversion on the detection signal corresponding to the electrocardiographic waveform amplified by the amplifier unit 102, and outputs the resultant signal to the control unit 104.

The control unit 104 is in charge of controlling the portable electrocardiographic device 1, and includes, for example, a central processing unit (CPU) or the like. Upon receiving an electrocardiographic waveform measurement start instruction from the subject through the operation unit 108, the control unit 104 records, in the memory unit 106, measurement data related to the electrocardiographic waveform within a predetermined period detected by the electrode unit 101. The control unit 104 analyzes the electrocardiographic waveform detected within the predetermined period, and stores the analysis result in the memory unit 106. When the abnormal waveform is detected as a result of the electrocardiographic waveform analysis, the control unit 104 turns on the abnormal waveform detection LED 13. Furthermore, the operation unit 108 controls the components of the portable electrocardiographic device 1 to execute processing corresponding to an operation performed on the operation unit 108 by the subject.

The data on the electrocardiographic waveform measured within the predetermined period and the result of analysis on the electrocardiographic waveform, recorded in the memory unit 106, are provided to a cooperating information processing device such as a smartphone or a PC through BLE communications or the like. In medical institutions, heart disease can be detected at an early timing and appropriate treatment can be performed, based on the measurement data on the electrocardiographic waveform, the analysis result, and the like provided from the portable electrocardiographic device 1.

The timer unit 105 receives an instruction from the control unit 104, and counts various periods related to the electrocardiographic waveform measurement. The memory unit 106 is configured to include a main memory such as a read only memory (ROM) and a random access memory (RAM) as well as, for example, a long-term storage medium such as flash memory. The memory unit 106 stores various programs related to electrocardiographic waveform measurement and analysis, and various types of information for detecting an abnormal waveform and the like. The display unit 107 displays various types of information related to the electrocardiographic waveform measurement. The display unit 107 includes the power source LED 7, the BLE communication LED 9, the memory residual display LED 10, the battery exchange LED 11, the measurement notification LED 12, and the abnormal waveform detection LED 13. The display unit 107 may also include a liquid crystal display unit, a speaker for notifying the subject of a message using sounds, and the like.

The operation unit 108 receives an operation input from the subject. The operation unit 108 includes the power switch 6 and the BLE communication button 8. The power source unit 109 supplies power for making the portable electrocardiographic device 1 function, and includes a battery, a secondary battery, and the like. The communication unit 110 is a communication interface that is in charge of transmission and reception of a signal to and from a device such as a smartphone. The communication function provided by the communication unit 110 may be BLE communications, for example, but other known wireless and wired communication schemes can be employed.

Electrocardiographic Waveform Measurement Processing

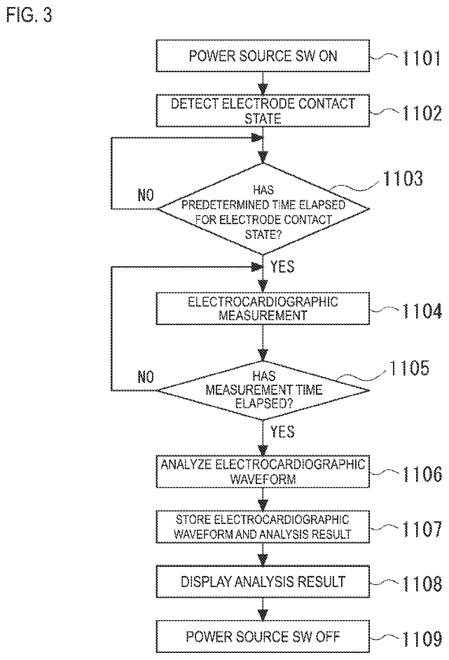

FIG. 3 is a flowchart illustrating an example of electrocardiographic waveform measurement processing in the portable electrocardiographic device 1 according to the present embodiment. FIG. 3 illustrates an example of processing in a case where the electrocardiographic waveform is measured by the portable electrocardiographic device 1 alone. The flow is implemented by the control unit 104 of the portable electrocardiographic device 1 executing a program stored in the memory unit 106.

An example of a timing when the processing in the flow starts includes a timing when an operation of pressing the power switch 6, provided on the left side surface of the main body 1a, by the subject is received (step 1101). When the operation of pressing the power switch 6 is received, the power source LED 7 is turned on and the processing proceeds to step 1102. In step 1102, an electrode contact state that is a state of contact with the electrode (the electrode a 2, the electrode b 3, and the electrode c 4) related to the electrocardiographic waveform measurement is detected. For example, a relative potential difference, from the reference potential detected with the earth electrode (electrode b 3), is detected between the pair of measurement electrodes (the electrode a 2 and the electrode c 4), and the processing proceeds to step 1103. In step 1103, it is determined whether a predetermined period of time has elapsed with the electrode contact state detected by the electrode related to the electrocardiographic waveform measurement maintained. Whether the predetermined period of time has elapsed is determined based on time measured using the timer unit 105. When it is determined that the predetermined period of time has elapsed in step 1103 (“Yes” in step 1103), the processing proceeds to step 1104. On the other hand, when it is determined that the predetermined period of time has not elapsed yet, the processing proceeds to step 1103 (“No” in step 1103).

In step 1104, the electrocardiographic waveform measurement for the subject starts. When the electrocardiographic waveform measurement starts, the measurement notification LED 12 provided on the main body 1a is turned on or blinks, to notify the subject of the portable electrocardiographic device 1 being currently in the electrocardiographic waveform measurement state. When the electrocardiographic waveform measurement starts, data on the electrocardiographic waveform measured is recorded in the memory unit 106 together with time information. After step 1104, the processing proceeds to step 1105.

In step 1105, it is determined whether measurement time has elapsed. Whether the measurement time has elapsed is determined based on time measured using the timer unit 105. When it is determined that the measurement time has elapsed in step 1105 (“Yes” in step 1105), the processing proceeds to step 1106. On the other hand, when it is determined that the measurement time has not elapsed yet (“No” in step 1105), the processing returns to step 1104 and the electrocardiographic waveform measurement continues.

In step 1106, the electrocardiographic waveform of the subject is analyzed based on the data on the electrocardiographic waveform measured. The electrocardiographic waveform is analyzed based on time series data recorded in the memory unit 106. After the processing in step 1106, the data on the measured electrocardiographic waveform, and the analysis result are stored in the memory unit 106 in step 1107. For example, the data and the analysis result of the electrocardiographic waveform recorded in the RAM and the like of the memory unit 106 are associated with each other, and stored in a long-term storage medium such as flash memory. When in step 1108, after the processing in step 1107, an abnormal waveform is detected as a result of the electrocardiographic waveform analysis, the abnormal waveform detection LED 13 provided to the main body 1a is turned on. With the abnormal waveform detection LED 13 turned on, the subject is notified of the electrocardiographic waveform measured including the abnormal waveform. After the processing in step 1108, the processing proceeds to step 1109 in which an operation of turning off the power switch 6 by the subject is received. When the operation of turning off the power switch 6 after the end of the measurement is performed, the portable electrocardiographic device 1 stops the power supply from the power source unit 109, and turns off the power source LED 7.

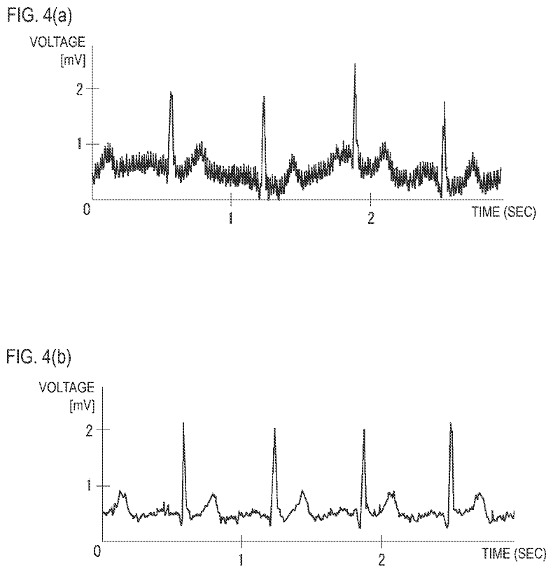

FIGS. 4(a) and 4(b) are diagrams illustrating examples of an electrocardiographic waveform measured by the portable electrocardiographic device 1 according to the first embodiment. FIGS. 4(a) and 4(b) illustrate examples of graphs of electrocardiographic waveforms, with the vertical axis representing a voltage value (mv) and the horizontal axis representing elapsed time (seconds). FIG. 4(a) illustrates an example of a graph of an electrocardiographic waveform measured with an insufficient state of contact with the measurement electrode that is the electrode c 4. FIG. 4(b) illustrates an example of a graph of an electrocardiographic waveform measured by the portable electrocardiographic device 1 according to the present embodiment.

In the graph of the measured electrocardiographic waveform as illustrated in FIG. 4(a), noise is superimposed on the lower potential side due to the insufficient contact to the measurement electrode c 4. In addition, noise influence affecting even a peak value of the electrocardiographic waveform makes the voltage level unstable, and thus fluctuation can be seen. On the other hand, in the present embodiment, the proximal end side of the right hand forefinger is in close contact with the measurement electrode c 4 during the electrocardiographic waveform measurement, and thus the finger and the electrode, related to the electrocardiographic waveform measurement, are in a stable contact state. Thus, it can be seen that the mixing of noise on the lower potential side is suppressed. Furthermore, the influence of noise on the peak value of the electrocardiographic waveform is suppressed. Thus, a relative variation amount (relative variation range of the voltage level between peak values) with respect to the peak value is reduced. Thus, it can be seen that the voltage level is stable.

As described above, the portable electrocardiographic device 1 according to the present embodiment is configured to have the electrode a 2 provided on the bottom portion of the main body 1a and the electrode c 4 provided on the right side of the upper portion of the main body 1a forming a pair of measurement electrodes. The electrode b 3 provided on the left side of the upper portion of the main body 1a is configured as an earth electrode for finding a reference potential for a change in potential of the body. As a result, the electrocardiographic waveform can be measured with the proximal end side of the right hand forefinger being in close contact with the electrode c 4 serving as the measurement electrode for finding a change in body potential. Thus, the stable contact state between the finger and the electrode is achieved, whereby the accuracy of the electrocardiographic waveform measurement can be improved.

### Second Embodiment

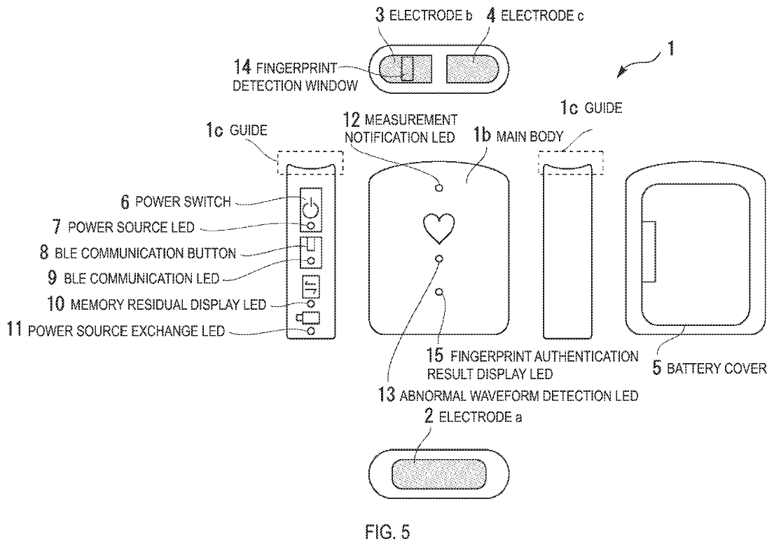

Next, another example of the embodiments of the present invention will be described based on FIGS. 5 to 8. It should be noted that the dimension, material, shape, relative arrangement and the like of the components described in the present embodiment are not intended to limit the scope of this invention to them alone, unless otherwise stated. Configurations that are the same as those in the first embodiment are denoted by the same reference numerals, and the detailed description thereof will be omitted. A difference from the first embodiment is mainly described.

Device Configuration of Second Embodiment

FIG. 5 is a six-view diagram illustrating an example of a configuration of a portable electrocardiographic device according to the present embodiment. The portable electrocardiographic device 1 according to the present embodiment includes the electrode a 2, the electrode b 3, and the electrode c 4 for measuring an electrocardiographic waveform of a subject, as in the first embodiment. As illustrated in FIG. 5, the electrode b 3 is provided with a fingerprint detection window 14 so that a fingerprint on the distal end side of the right hand forefinger of the subject can be detected by a fingerprint sensor. The fingerprint detection window 14 is formed by, for example, a transparent electrode member (conductive member) that is transparent.

The disposed positions of the electrodes in the present embodiment are the same as those in the first embodiment. Specifically, the electrode a 2 is provided on the bottom portion side of a main body 1b, the electrode b 3 is provided on the upper portion left side of the main body 1b, and the electrode c 4 is provided on the upper portion right side of the main body 1b. The electrode a 2 and the electrode c 4 form a pair of measurement electrodes, the electrode b 3 provided on the left side of the upper portion of the main body 1b is configured as an earth electrode for finding a reference potential for a change in potential of a body. The upper portion side of the main body 1b, where the electrode b 3 and the electrode c 4 are disposed, is processed to be in a recess form (guide 1c) to facilitate close contact of the right hand forefinger, as indicated by a rectangular region surrounded by a dashed line.

When the lead I measurement is performed for the electrocardiographic measurement, the portable electrocardiographic device 1 can be held with the distal end side, of the right hand forefinger bent in a substantially L shape along the curved shape on the upper portion side of the main body 1b, brought in close contact with the electrode b 3 on the left side, and with the proximal end side brought in contact with the electrode c 4 on the right side. The same applies to the case where the lead V4 measurement is performed for the electrocardiographic measurement. Also in the present embodiment, the electrode b 3 serving as the earth electrode can be disposed at the position to be in contact with the distal end side of the right hand forefinger, and the electrode c 4 serving as the negative electrode of the measurement electrodes can be disposed at the position to be in contact with the proximal end side, whereby the electrocardiographic measurement can be performed with the finger and the electrode being in a stable contact state.

With the portable electrocardiographic device 1 according to the present embodiment, fingerprint detection is performed on the distal end side of the right hand forefinger in close contact with the electrode b 3, during electrocardiographic measurement, using the fingerprint detection window 14 provided to the electrode b 3. With the portable electrocardiographic device 1, the fingerprint detected using the fingerprint detection window 14 is compared with the fingerprint of the forefinger on the distal end side of the right hand forefinger of the subject registered in advance, to prevent the finger from being in contact with the electrode b 3 in a wrong direction when the electrocardiographic waveform is measured.

For example, when the fingerprint registered in advance and the fingerprint detected using the fingerprint detection window 14 match and a predetermined condition is satisfied, the direction of the right hand forefinger in contact with the upper portion of the main body 1b can be determined to be appropriate. On the other hand, when the fingerprint registered in advance and the fingerprint detected using the fingerprint detection window 14 do not match and the predetermined condition fails to be satisfied, the direction of the right hand forefinger in contact with the upper portion of the main body 1b can be determined to be inappropriate. An example of the inappropriate state is a case where the distal end side of the right hand forefinger is in contact with the electrode c 4 serving as the negative electrode of the measurement electrodes and the proximal end side is in contact with the electrode b 3 serving as the earth electrode for example. In such an inappropriate state, the contact between the forefinger and the measurement electrode is likely to be unstable, for example. When force is applied to the fingertip to make the contact, electromyographic noise is superimposed, and accurate electrocardiographic waveform measurement is hindered. In the present embodiment, based on the result of the comparison with the fingerprint on the distal end side of the right hand forefinger of the subject registered in advance, an inappropriate used state is determined, whereby the accuracy of the electrocardiographic waveform measurement can be improved. The subject is notified of the result of the authentication of the fingerprint detected using the fingerprint detection window 14, with a fingerprint authentication result display LED 15 of the main body 1b turned on.

System Configuration of Second Embodiment

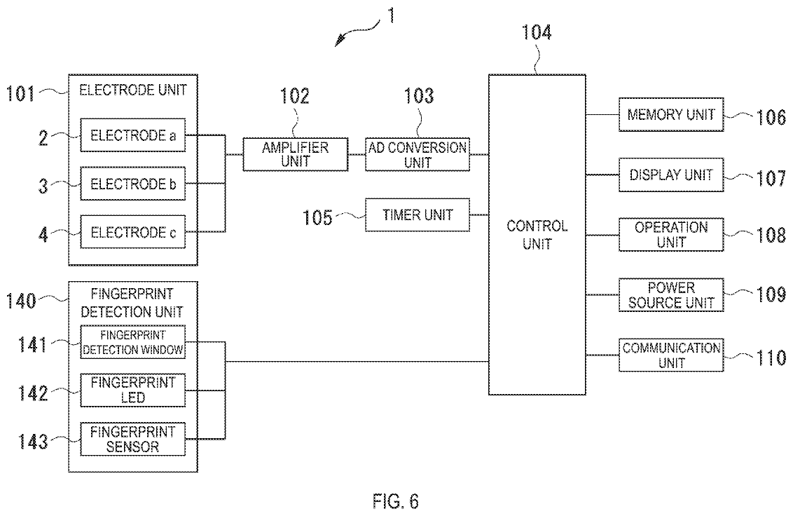

FIG. 6 is a diagram illustrating an example of a system configuration of the portable electrocardiographic device 1 according to the present embodiment. As illustrated in FIG. 6, the portable electrocardiographic device 1 includes a fingerprint detection unit 140, in addition to the configuration of the first embodiment. The display unit 107 includes the fingerprint authentication result display LED 15 provided to the main body 1b. In the memory unit 106, information on the fingerprint on the distal end side of the right hand forefinger of the subject measured in advance is registered.

The fingerprint detection unit 140 is configured to include a fingerprint detection window 141, a fingerprint LED 142, and a fingerprint sensor 143. The fingerprint detection unit 140 is connected to the control unit 104. The fingerprint detection window 141 is the fingerprint detection window 14 provided to the electrode b 3, and is formed of a transparent electrode member. The fingerprint LED 142 is a light emitting element related to fingerprint comparison. The fingerprint sensor 143 is a sensor for detecting a fingerprint at a site in contact with the fingerprint detection window 14, at the start of measurement of the electrocardiographic waveform. While an optical scheme is described as an example of the fingerprint comparison scheme in FIG. 6, other fingerprint comparison schemes such as an ultrasonic scheme or an electrostatic capacitive type may be employed for example. In such a case, the fingerprint detection unit 140 is configured based on the type of the fingerprint comparison scheme. The fingerprint information detected by the fingerprint detection unit 140 is output to the control unit 104. The control unit 104 compares the fingerprint information detected by the fingerprint detection unit 140 with the fingerprint information registered in the memory unit 106, and determines whether the fingerprint authentication is successful as the result of the comparison.

Processing Flow

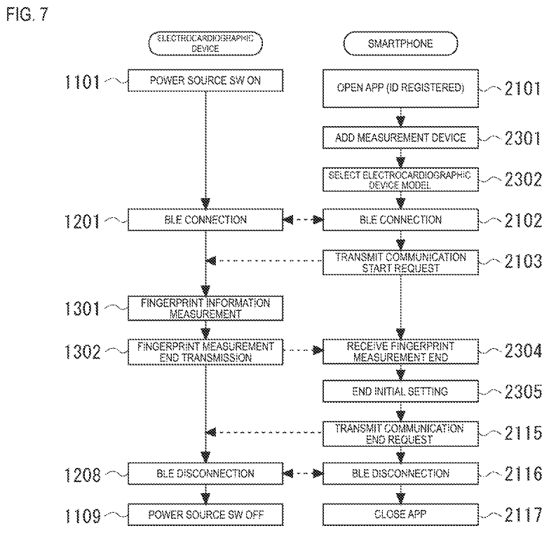

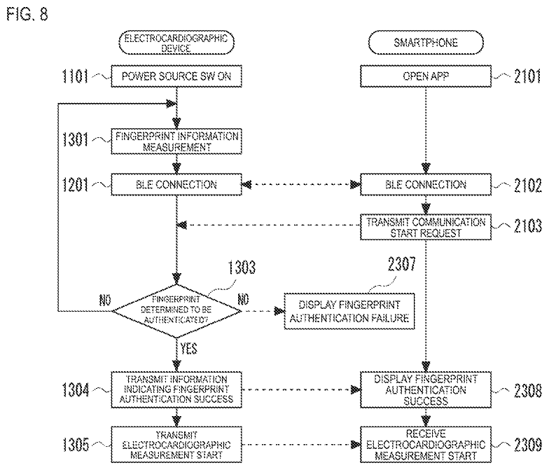

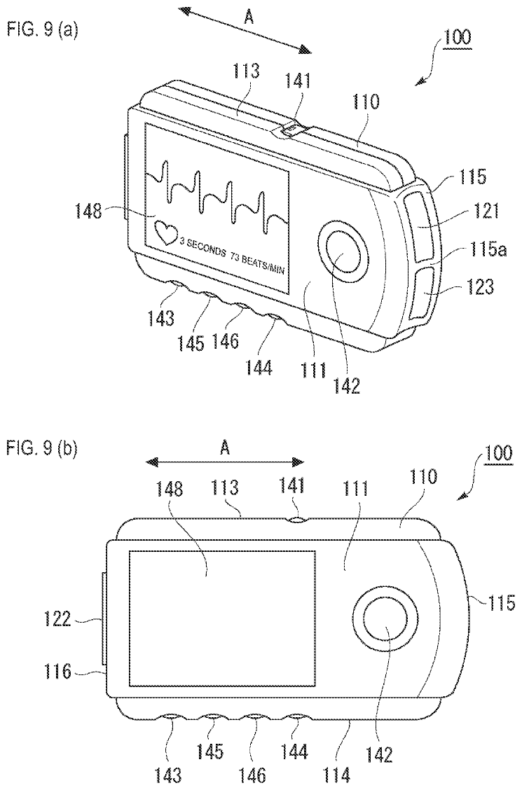

Next, with reference to FIGS. 7 and 8, a flow of processing using fingerprint information in the portable electrocardiographic device 1 according to the present embodiment is described. FIG. 7 is a flowchart illustrating an example of processing related to an initial setting using fingerprint information between the portable electrocardiographic device 1 and a cooperating information processing device such as a smartphone. In the information processing device such as a smartphone that cooperates with the portable electrocardiographic device 1, an application program (hereinafter, also referred to as an application) is installed that can collect information measured by various biological information measurement devices other than the electrocardiographic device, for example, a sphygmomanometer, a body composition meter, and an activity meter. Through the processing in FIG. 7, the initial setting for the cooperating smartphone or the like is performed so that the electrocardiographic waveform measured using the portable electrocardiographic device 1 as well as the analysis result can be collected.

In this flow, the portable electrocardiographic device 1 turns on the power source LED 7 upon receiving the operation of pressing the power switch 6, provided on the left side surface of the main body 1b, by the subject in step 1101, and the processing proceeds to step 1201. In step 1201, the portable electrocardiographic device 1 receives an operation of pressing the BLE communication button 8, provided on the left side surface of the main body 1b, by the subject, and then stands by until a communication start request is received from the cooperating smartphone or the like.

In the cooperating smartphone or the like, the application starts for collecting information measured by each biological information measurement device (such as electrocardiographic waveform, blood pressure value, body composition value, and activity value) through authentication using identification information (ID) of a user registered in advance (step 2101). When the application starts, a selection screen is displayed for adding a biological information measurement device that is the target of the information collection. When an operator of the smartphone or the like selects and adds a type (the electrocardiographic device, for example) of measurement equipment to be the target, from the various biological information measurement devices displayed on the selection screen (step 2301), the processing proceeds to step 2302. When the model for measuring an electrocardiographic waveform is further selected in step 2302 from the types of the measurement equipment added, the processing proceeds to step 2102. In step 2102, the BLE connection with the portable electrocardiographic device 1 selected in step 2302 is established, and the communication start request is transmitted (step 2103). After the end of step 2103, the smartphone or the like stands by until a notification indicating the end of the fingerprint information measurement is received from the measurement device that is the information collection target.

Upon receiving the communication start request transmitted from the cooperating smartphone or the like, the portable electrocardiographic device 1 turns on the BLE communication LED 9, and the processing proceeds to step 1301. In step 1301, fingerprint information on the distal end side of the right hand forefinger in contact with the electrode b 3 provided with the fingerprint detection window 14 is measured. The control unit 104 stores, in the memory unit 106, fingerprint information detected by the fingerprint sensor 143 forming the fingerprint detection unit 140. After the end of step 1301, the processing proceeds to step 1302, where the notification indicating the end of the fingerprint information measurement is transmitted to the cooperating smartphone or the like. After the end of step 1302, the portable electrocardiographic device 1 stands by until a communication end request transmitted from the cooperating smartphone or the like is received.

Upon receiving the notification indicating the end of the fingerprint measurement transmitted from the portable electrocardiographic device 1 in step 2304, the cooperating smartphone or the like ends the initial setting for the information collection (step 2305). Then, the smartphone or the like transmits the communication end request to the portable electrocardiographic device 1 connected thereto through the BLE communications (step 2115). After the end of step 2115, the communication connection with the portable electrocardiographic device 1 is disconnected (step 2116), and the application for the initial setting ends temporarily (step 2117).

Upon receiving the communication end request transmitted from the cooperating smartphone or the like, the portable electrocardiographic device 1 disconnects the BLE communication connection (step 1208). After the end of step 1208, the BLE communication LED 9 is turned off and the operation of turning off the power switch 6 performed by the subject is received (step 1109). When the operation of turning off the power switch 6 is performed, the portable electrocardiographic device 1 stops the power supply from the power source unit 109, and turns off the power source LED 7.

Next, a description is given with reference to FIG. 8. FIG. 8 is a flowchart illustrating an example of processing related to electrocardiographic waveform measurement using fingerprint information between the portable electrocardiographic device 1 and a cooperating information processing device such as a smartphone.

In this flow, the portable electrocardiographic device 1 turns on the power source LED 7 upon receiving the operation of pressing the power switch 6, provided on the left side surface of the main body 1b, by the subject in step 1101, and the processing proceeds to step 1301. In step 1301, fingerprint information on the distal end side of the right hand forefinger in contact with the electrode b 3 provided with the fingerprint detection window 14 is measured. The control unit 104 stores fingerprint information, detected by the fingerprint sensor 143 forming the fingerprint detection unit 140, in the memory unit 106. Then, the processing proceeds to step 1201.

In step 1201, the portable electrocardiographic device 1 receives an operation of pressing the BLE communication button 8, provided on the left side surface of the main body 1b, by the subject, and then stands by until a communication start request is received from the cooperating smartphone or the like.

In the cooperating smartphone or the like, the application starts for collecting information measured by each biological information measurement device (such as electrocardiographic waveform, blood pressure value, body composition value, and activity value) through authentication using identification information (ID) of a user registered in advance (step 2101). In step 2102, the BLE connection with the portable electrocardiographic device 1 that is the collection target starts, and the communication start request is transmitted (step 2103). After the end of step 2103, the smartphone or the like stands by until a notification indicating the successful fingerprint authentication is received from the portable electrocardiographic device 1 that is the collection target.

Upon receiving the communication start request transmitted from the cooperating smartphone or the like, the portable electrocardiographic device 1 turns on the BLE communication LED 9, and the processing proceeds to step 1303. In step 1303, the determination for fingerprint authentication is made based on the fingerprint information measured in step 1301. For example, the measured fingerprint information and the fingerprint information registered in the memory unit 106 are compared, and whether a predetermined condition is satisfied is determined (fingerprint authentication determination). Based on a result of the comparison, for example, whether the direction of the right hand forefinger placed on the upper portion of the main body 1b is appropriate can be determined.

When it is determined that the result of the fingerprint authentication is successful in step 1303 (“Yes” in step 1303), the processing proceeds to step 1304. On the other hand, when it is determined that the authentication has failed (“No” in step 1303), the fingerprint authentication result display LED 15 of the main body 1b is turned on (step 2307), and the processing proceeds to step 1301. With the fingerprint authentication result display LED 15 turned on, the subject can visually recognize that the finger is inappropriately brought in contact with the electrode b 3.

In step 1303, a fingerprint authentication failure notification may be transmitted to the cooperating smartphone or the like. Upon receiving the fingerprint authentication failure notification transmitted from the portable electrocardiographic device 1, the smartphone or the like in the standby state can display a message to the subject. For example, a notification indicating a procedure required for accurately measuring the electrocardiographic waveform may be issued by means of a display screen of or a sound from the smartphone or the like. For example, it is possible to prompt the subject to bring the distal end side of the right hand forefinger in contact with the fingerprint detection window 14 provided to the electrode b 3, and to bring the proximal end side of the right hand forefinger in contact with the electrode c 4.

In step 1304, a fingerprint authentication success notification is transmitted to the cooperating smartphone or the like. Then, the processing proceeds to step 1305, where the notification indicating the start of the electrocardiographic waveform measurement for the subject is transmitted to the cooperating smartphone or the like.

After the end of the processing in step 1305, the electrocardiographic waveform measurement processing described with reference to FIG. 3 starts, for example.

Upon receiving the fingerprint authentication success notification transmitted from the portable electrocardiographic device 1, the cooperating smartphone or the like notifies the subject of the current contact state of the right hand forefinger related to the electrocardiographic waveform measurement being appropriate in step 2308. For example, the notification described above can be issued to the subject, by means of the display screen, the speaker, or the like of the smartphone or the like. After the end of step 2308, the processing proceeds to step 2309, where the electrocardiographic waveform measurement start notification transmitted from the portable electrocardiographic device 1 is received.

As described above, the portable electrocardiographic device 1 according to the present embodiment is configured with the fingerprint detection window 14 provided to the electrode b 3, serving as the earth electrode for finding the reference potential for a change in potential of the body. Furthermore, the configuration is employed to enable detection of the fingerprint on the distal end side of the right hand forefinger of the subject, by the fingerprint sensor using the fingerprint detection window 14 in the appropriate contact state. As a result, for the electrocardiographic measurement, the fingerprint detected using the fingerprint detection window 14 provided to the electrode b 3 can be compared with the fingerprint at a distal end portion of the right hand forefinger of the subject registered in advance. In the present embodiment, when the direction of the right hand forefinger in contact with the upper portion of the main body 1b is determined to be inappropriate, the subject can be notified of such a state, and prompted to perform the electrocardiographic waveform measurement under an appropriate contact state. With the present embodiment, the finger can be prevented from being in a wrong direction to be in contact with the electrode b 3 for measuring the electrocardiographic waveform, whereby the accuracy of the electrocardiographic waveform measurement can be improved.

### REFERENCE NUMERALS LIST

1 Portable electrocardiographic device
1a Main body
1b Main body
1c Guide
2 Electrode a
3 Electrode b
4 Electrode c
5 Battery cover
6 Power switch
7 Power source LED
8 BLE communication button
9 BLE communication LED
10 Memory residual display LED
11 Battery exchange LED
12 Measurement notification LED
13 Abnormal waveform detection LED
14 Fingerprint detection window
15 Fingerprint authentication result display LED
101 Electrode unit
102 Amplifier unit
103 AD conversion unit
104 Control unit
105 Timer unit
106 Memory unit
107 Display unit
108 Operation unit
109 Power source unit
110 Communication unit
140 Fingerprint detection unit
141 Fingerprint detection window
142 Fingerprint LED
143 Fingerprint Sensor

1. A portable electrocardiographic device comprising:
an electrode unit configured to detect an electrocardiographic waveform; and
a control unit configured to record, in a storage unit, the electrocardiographic waveform detected by the electrode unit, wherein
the electrode unit includes a pair of measurement electrodes for measuring the electrocardiographic waveform, and an earth electrode for finding a reference potential for a change in a potential of a body, and
the earth electrode is disposed in such a manner that a distal end side of a finger of a subject that is a target of the electrocardiographic waveform measurement is brought in contact with the earth electrode,
one of the measurement electrodes is disposed on a proximal end side of the finger of the subject, the distal end side of which is in contact with the earth electrode, and
the earth electrode disposed in such a manner that the distal end side of the finger of the subject is brought in contact with the earth electrode is provided with a fingerprint detection unit configured to detect a fingerprint of the finger in contact with the fingerprint detection unit.

2. The portable electrocardiographic device according to claim 1, wherein the distal end side includes a distal phalanx of the finger of the subject that is the target of the electrocardiographic waveform measurement, and the proximal end side includes a middle phalanx site of the finger, the distal end side of which is in contact with the earth electrode.

3. The portable electrocardiographic device according to claim 1, wherein a region, of the earth electrode, where the fingerprint detection unit is provided is formed by a transparent electrode.

4. The portable electrocardiographic device according to claim 1, wherein the earth electrode is provided with a fingerprint detection window for detecting a fingerprint of the finger in contact with the fingerprint detection window.

5. The portable electrocardiographic device according to claim 1, wherein the control unit records, in the storage unit, fingerprint information on the subject detected by the fingerprint detection unit provided to the earth electrode, and determines whether a result of comparison between registered fingerprint information registered in the storage unit and the fingerprint information on the subject satisfies a predetermined condition.

6. The portable electrocardiographic device according to claim 5, wherein the control unit starts measuring the electrocardiographic waveform of the subject, when the result of the comparison between the registered fingerprint information registered in the storage unit and the fingerprint information on the subject satisfies the predetermined condition.

7. The portable electrocardiographic device according to claim 5, wherein when the result of the comparison between the registered fingerprint information registered in the storage unit and the fingerprint information on the subject is determined to satisfy the predetermined condition, the control unit notifies the subject of fingerprint authentication using the fingerprint information on the subject having failed, or a contact state of the finger in contact with the earth electrode being inappropriate.

8. The portable electrocardiographic device according to claim 6, wherein when the result of the comparison between the registered fingerprint information registered in the storage unit and the fingerprint information on the subject is determined not to satisfy the predetermined condition, the control unit notifies the subject of fingerprint authentication using the fingerprint information on the subject having failed, or a contact state of the finger in contact with the earth electrode being inappropriate.
